(19) United States
(12) Patent Application Publication
(10) Pub. No.: US 2026/0360114 A1
(43) Pub. Date: Nov. 12, 2026

(54) IMAGE CLASSIFICATION DEVICE, METHOD, AND COMPUTER-READABLE STORAGE MEDIUM FOR ENDOSCOPIC IMAGES

(71) Applicant: OLYMPUS MEDICAL SYSTEMS CORP., Tokyo (JP)

(72) Inventors: Koichi SHINTANI, Tokyo (JP); Manabu ICHIKAWA, Tokyo (JP); Junichi ICHIKAWA, Tokyo (JP); Hiromu IKEDA, Tokyo (JP); Ryo INOMATA, Tokyo (JP); Osamu NONAKA, Tokyo (JP)

(73) Assignee: OLYMPUS MEDICAL SYSTEMS CORP., Tokyo (JP)

(21) Appl. No.: 19/720,634

(22) Filed: Jun. 25, 2026

Related U.S. Application Data
(63) Continuation of application No. PCT/JP2023/046630, filed on Dec. 26, 2023.

(51) Int. Cl. G06V 10/764; A61B 1/00; G06T 7/00; G06V 10/12; G06V 10/44; G06V 10/74; G06V 20/40; G16H 40/63

(52) U.S. Cl. CPC G06V 10/764; A61B 1/00006; A61B 1/000096; A61B 1/00045; G06T 7/0014; G06V 10/12; G06V 10/44; G06V 10/761; G06V 20/41; G06V 20/46; G16H 40/63; G06T 2207/10016; G06T 2207/10068; G06T 2207/30004; G06V 2201/03

(57) ABSTRACT

An image classification device for an endoscope includes a connection interface and processing circuitry. The processing circuitry is configured to obtain, via the connection interface, images sequentially captured in a process of insertion of the endoscope into a body of a subject or withdrawal of the endoscope from the body, and determine anatomical sites in the body through which the endoscope has passed, according to the obtained images. The processing circuitry is further configured to determine site-specific features of each of the determined anatomical sites for one or more determination items, thereby obtaining anatomical site-specific feature information for each of the determined anatomical sites, and record the anatomical site-specific feature information for each of the determined anatomical sites in association with the obtained images.

### CROSS REFERENCE TO RELATED APPLICATION

This application is a continuation of International Application No. PCT/JP2023/046630, filed on Dec. 26, 2023, the entire contents of which are incorporated herein by reference.

### FIELD

Aspects of the present disclosure relate to an image classification device for an endoscope, a method, and a non-transitory computer-readable storage medium, which are configured to classify and organize images, which are obtained in a process from insertion to withdrawal of the endoscope, for using the images for examination, diagnosis, etc.

### BACKGROUND

Endoscopes are devices that are configured to be inserted into a body, etc., to thereby enable observation of a diseased part and the like which cannot be seen from outside. Such an endoscope includes an insertion portion to be inserted into a body cavity, and includes an image pickup apparatus in a distal end of the insertion portion, for example. In an examination using such an endoscope, a physician sequentially displays images acquired by the image pickup apparatus in the distal end of the insertion portion, adjusts a position of the distal end of the insertion portion while confirming the displayed images, and diagnoses a health condition and a state of a disease.

In a process from insertion to withdrawal of such an insertion portion, the image pickup apparatus continues to pick up images, and in an endoscopy, information such as an enormous number of images of respective parts (organs) of a human body through which the insertion portion passed is acquired in addition to the images to be used for diagnosis and the like.

Japanese Patent Application Laid-Open Publication No. 2002-253539 (hereinafter, referred to as Patent Literature 1) proposes a technology for managing acquired image information. This proposal discloses a technology of identifying a medical image and adding thereto, as management information, image pickup attribute information of a category which is most coincident with a category of the identified medical image.

### SUMMARY

According to aspects of the present disclosure, an image classification device for an endoscope is provided. The image classification device includes a connection interface and processing circuitry. The processing circuitry is configured to obtain, via the connection interface, images sequentially captured in a process of insertion of the endoscope into a body of a subject or withdrawal of the endoscope from the body. The processing circuitry is further configured to determine anatomical sites in the body through which the endoscope has passed, according to the obtained images. The processing circuitry is further configured to determine site-specific features of each of the determined anatomical sites for one or more determination items, thereby obtaining anatomical site-specific feature information for each of the determined anatomical sites. The processing circuitry is further configured to record the anatomical site-specific feature information for each of the determined anatomical sites in association with the obtained images.

According to aspects of the present disclosure, further provided is a method implemented by processing circuitry. The method includes obtaining images sequentially captured in a process of insertion of an endoscope into a body of a subject or withdrawal of the endoscope from the body. The method further includes determining anatomical sites in the body through which the endoscope has passed, according to the obtained images. The method further includes determining site-specific features of each of the determined anatomical sites for one or more determination items, thereby obtaining anatomical site-specific feature information for each of the determined anatomical sites. The method further includes performing at least one of recording the anatomical site-specific feature information for each of the determined anatomical sites in association with the obtained images, or searching for endoscopic images obtained in a second case using the anatomical site-specific feature information obtained for each of the determined anatomical sites in a first case.

According to aspects of the present disclosure, further provided is a non-transitory computer-readable storage medium storing computer-readable instructions. The computer-readable instructions are configured to, when executed by processing circuitry, cause the processing circuitry to obtain images sequentially captured in a process of insertion of an endoscope into a body of a subject or withdrawal of the endoscope from the body. The computer-readable instructions are further configured to, when executed by processing circuitry, cause the processing circuitry to determine anatomical sites in the body through which the endoscope has passed, according to the obtained images. The computer-readable instructions are further configured to, when executed by processing circuitry, cause the processing circuitry to determine site-specific features of each of the determined anatomical sites for one or more determination items, thereby obtaining anatomical site-specific feature information for each of the determined anatomical sites. The computer-readable instructions are further configured to, when executed by processing circuitry, cause the processing circuitry to perform at least one of recording the anatomical site-specific feature information for each of the determined anatomical sites in association with the obtained images, or searching for endoscopic images obtained in a second case using the anatomical site-specific feature information obtained for each of the determined anatomical sites in a first case.

### BRIEF DESCRIPTION OF DRAWINGS

FIG. 1 is a block diagram showing an image classification device for an endoscope according to an embodiment of the present disclosure.

FIG. 2 is an explanatory diagram showing an example of an input image.

FIG. 3 is an explanatory diagram for explaining an inference model for enabling site determination.

FIG. 4 is an explanatory diagram showing an example summarizing correspondences between respective sites and respective determination items in a table format.

FIG. 5 is an explanatory diagram showing examples of a shape of a large intestine.

FIG. 6 is a flowchart for explaining an operation in the embodiment.

FIG. 7 is a flowchart for explaining the operation in the embodiment.

FIG. 8 is an explanatory diagram for explaining an example of an endoscopy.

FIG. 9 is an explanatory diagram for explaining images acquired in the example in FIG. 8.

FIG. 10 is a flowchart for explaining the operation in the embodiment.

### DESCRIPTION OF EMBODIMENTS

Patent Literature 1 proposes a technology of automatically adding image pickup attribute information, such as an image pickup apparatus, an image pickup site, and an image pickup direction, to a medical image, as management information. With the device in Patent Literature 1, it is easy to distinguish from which direction, an image of which site is picked up using what kind of devices, for example, among an X-ray device, an MRI device, and the like. For example, it is easy to distinguish whether an image of a head part is picked up or an image of a chest part is picked up. The technology in Patent Literature 1 enables discrimination of images of which of the sites, and facilitates searches of past cases of a specific examinee. However, in the proposal of Patent Literature 1, the acquired management information is effective for the diagnosis of the examinee, himself or herself, while in a case where the acquired management information is used as reference information or the like for diagnosis of another person, a troublesome search operation is required. According to the present disclosure, an image classification device for an endoscope, an image classification method, an image classification program, and a search method are configured to accumulate medical information acquired in a process from insertion to withdrawal of the endoscope, and by performing classification according to a feature of each of the sites based on images of each of the sites, it is possible to obtain information extremely useful for diagnosis and treatment.

Hereinafter an embodiment of the present disclosure will be described in detail with reference to drawings.

### Embodiment

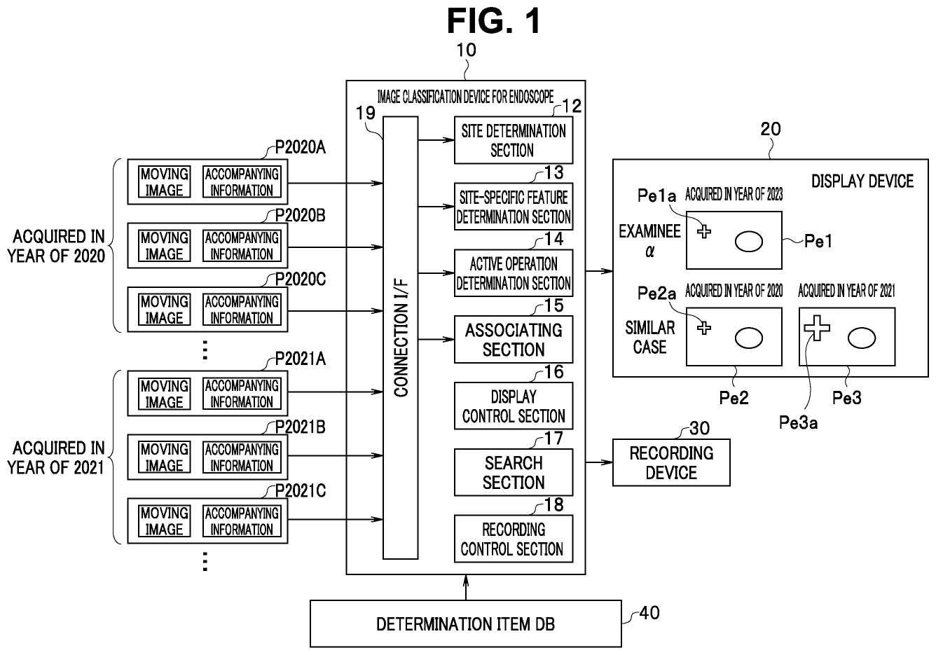

FIG. 1 is a block diagram showing an image classification device for an endoscope according to an embodiment of the present disclosure. A moving image obtained by an endoscopy includes images of various sites from outside a body to various organs such as a throat, an esophagus, a stomach, a duodenum, and is rich in information, but it is not fully used. In the present embodiment, respective sites are classified according to a plurality of features based on the images of the respective sites, classification results are accumulated, to enable the accumulated classification results to be used as information useful for diagnosis. For example, classifying digestive organs by each of the features and accumulating the classification results enable determination of the features of the digestive organs of a person susceptible to diseases of the digestive organs, which can be used for diagnosis and prevention.

Note that the sites are anatomical sites, specific anatomical features of which can be identified. A unit of the sites may be a unit of an organ having a specific function. Note that the organs, such as a large intestine and a stomach, having a large volume, have names further subdivided medically (the stomach includes a cardia, a fornix, a stomach body, an antrum, a pylorus, etc., and the large intestine includes an ascending colon, a transverse colon, a descending colon, a sigmoid colon, a rectum, etc.). Therefore, a subdivided part may be referred to as a site, or a subdivided part of an organ, which is subdivided in a method different from a medical subdividing method, may be referred to as a site.

In FIG. 1, medical images such as endoscopic images acquired by an endoscope, etc., not shown, are inputted to an image classification device 10 for an endoscope (hereinafter simply referred to as the image classification device 10). The endoscope includes an insertion portion configured to be inserted into a body cavity and an image pickup apparatus provided in a distal end of the insertion portion. The image pickup apparatus includes, for example, an image pickup device such as a CCD or a CMOS sensor, and photoelectrically converts an optical image from a photographic object, to obtain an image pickup signal. There is a case where the image pickup signal is supplied to a video processor, not shown, to be subjected to signal processing, and then supplied to the image classification device 10. In addition, a channel (a communication passage that connects an inside of a body cavity and an outside of a body) through which an instrument such as a treatment instrument is passed is provided inside the endoscope along a tube of the endoscope. Through such a channel, an ultrasonic sensor and the like can be used, and various sensors and a transmitter can be arranged together in a distal end portion of the endoscope. In cooperation with such sensors or other instruments, information other than images can also be acquired according to insertion of the endoscope into the body. The cooperating device may be an in-hospital system, and the like, and there is a case where voices, comments, and a terminal operation of a physician during an endoscopy can also be used. Since moving image information of the endoscope is time passage information over time, useful information may be made to be available in a manner corresponding to timings of the moving image. Such information may be recorded in association with images, as “accompanying information” in FIG. 1. The image classification device 10 is described as a special device that classifies the images obtained from the multiple endoscopes, but it may classify the results of the endoscopy in real time by directly connecting the endoscope thereto, and may, in cooperation with a display control section 16, give a guide applying the functions thereof, in an endoscopy.

The medical images inputted to the image classification device 10 are images of an inside of a body, which are acquired in the endoscopy and the like. The input images, for example, the endoscopic images, may be directly supplied from the video processor, or may be images, which are recorded in a recording device, read out and supplied. FIG. 1 shows an example of moving images as the images (input images) inputted to the image classification device 10, but not only a series of images acquired continuously but also still images, which are acquired as needed, by an operation of a medical professional or under specific conditions, may be inputted.

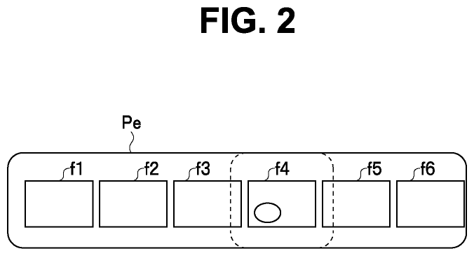

FIG. 2 is an explanatory diagram showing an example of the input images. The quadrangular frames in FIG. 2 indicate respective frames f1 to f6 of a series of images Pe that are acquired continuously. The frame f4 includes an image part of a lesion part shown by an ellipse. In a normal endoscopy, the image in the frame f4 is used for diagnosis, and the like, and generally, the images in the other frames f1 to f3, f5, and f6 are not used for any purpose other than insertion of the endoscope.

FIG. 1 shows an example in which multiple images including moving images of three persons A to C are inputted. The images acquired for the persons A to C in the year of 2020 are input images P2020A to P2020C, respectively, and the images acquired for the persons A to C in the year of 2021 are input images P2021A to P2021C, respectively.

Note that the input images may be added with accompanying information, in addition to the moving images. The accompanying information includes operation information, sensor information, setting information of endoscope instruments, information regarding actions of a physician who performs the endoscopy and an assistant during the endoscopy, information regarding used treatment instruments, and other information such as a diagnosis result, pathological findings, a consultation result, etc.

The accompanying information may include information regarding the hospital, information regarding the operation examination room, the date and time of the operation examination, patient information such as age, race, gender, etc., information regarding the physician in charge and the medical professionals participated in the examination, etc. The accompanying information may include information regarding other instruments in the examination. For example, in recent years, since conversations between physicians and medical professionals can be collected by a microphone and recorded, such information can be recorded so as to correspond to the image frames.

Similarly, the accompanying information may include the above-described information regarding the various sensors. For example, various switches are provided to an operation portion of the endoscope not shown. In some cases, operation information regarding the operations and the like of the switches may be inputted as being added, as the accompanying information, to image data of endoscopic images. In addition, in other cases, operation information based on an operation of a gas/liquid feeding device is added as the accompanying information. If such operation information includes information indicating that it corresponds to which timing of the moving image frames, the operation information can be used for the image classification for endoscope in the present application.

In addition, in a case of a special light observation, a special light observation image has features different from those of a normal image. In view of this, it is sometimes better to handle the special light observation image separately from a normal light observation image, for various kinds of determination. Accordingly, information for discriminating whether respective frames of picked-up images are the special light observation images or the normal light observation images may be added as accompanying information to image information. In addition, information acquired by an endoscope insertion shape observation device may be added as the accompanying information to the image information.

The image classification device 10 may be implemented by processing circuitry including one or more processors and non-transitory computer-readable storage media. The image classification device 10 includes: a site determination section 12, a site-specific feature determination section 13, an active operation determination section 14, an associating section 15, a display control section 16, a search section 17, and a recording control section 18. The constituent elements of the image classification device 10 may be implemented by processing circuitry including one or more processors using, for instance, a central processing unit (CPU) and a field programmable gate array (FPGA), a graphics processing unit (GPU), and the like, and may be configured to operate by one or more processors executing one or more programs stored in one or more non-transitory computer-readable storage media, to control respective sections, or may achieve at least one of the functions of the respective sections by a hardware electronic circuit. The image classification device 10 may further include a connection interface (hereinafter also referred to as a connection I/F) 19 configured to receive image data and information from external devices. The connection I/F 19 may be implemented as a wired and/or wireless interface, and may include, for instance, a communication interface, an input port, and/or a data acquisition interface.

In the present embodiment, the site determination section 12 of the image classification device 10 sequentially determines that the input images are the picked-up images of which of the sites. The site determination section 12 determines each of the sites in a body (e.g., human body) of an examinee, based on the endoscopic images sequentially acquired by the endoscope in an insertion process and a withdrawal process of the endoscope, that is, during a period from the start of the insertion to the completion of the withdrawal of the endoscope. The site determination section 12 not only can directly identify an anatomical feature of each of the sites from the images of each of the sites, but also, in some cases, can determine each of the sites also in a case where the anatomical feature of each of the sites can be analogized, based on the information of other respective sites, the anatomical features of which have been identified.

The site determination section 12, for example, determines the sites by an image analysis and inference processing of the endoscopic images. The site determination section 12, for example, can specify specific sites such as a nasal cavity, a stomach, etc., by determining the shape of the lumen from the image features. In addition, the site determination section 12, for example, distinguishes the advancing of the insertion portion while being bent based on the feature changes in the images (history of the images), to thereby be capable of determining the specific site such as the large intestine, and also determining whether the specific site is the descending colon, the transverse colon, or the like.

In addition, the site determination section 12 may determine the color, the shape, and patterns such as the blood vessels visible on the surface of the site, which are characteristic to the site, with reference to a database, not shown. Furthermore, there are other determination methods, such as a method of receiving, with an external receiver, a transmission result such as a magnetism by a transmitter provided in the distal end or the insertion portion of the endoscope to determine at which position in the body the distal end portion of the endoscope is located, thereby determining that the images are obtained as a result of image pickup of which of the sites, and a method of determining the sites based on an insertion amount and a degree of twisting of the endoscope. Representative images (images whose image qualities and the like have been adjusted, such as the images published in papers) of the respective sites and data indicating the image features of the respective sites may be recorded in a recording medium, not shown, and the site determination section 12 may determine the respective sites while referring to the information recorded in the recording medium.

In addition, the site determination section 12 may determine the sites by artificial intelligence (AI) processing.

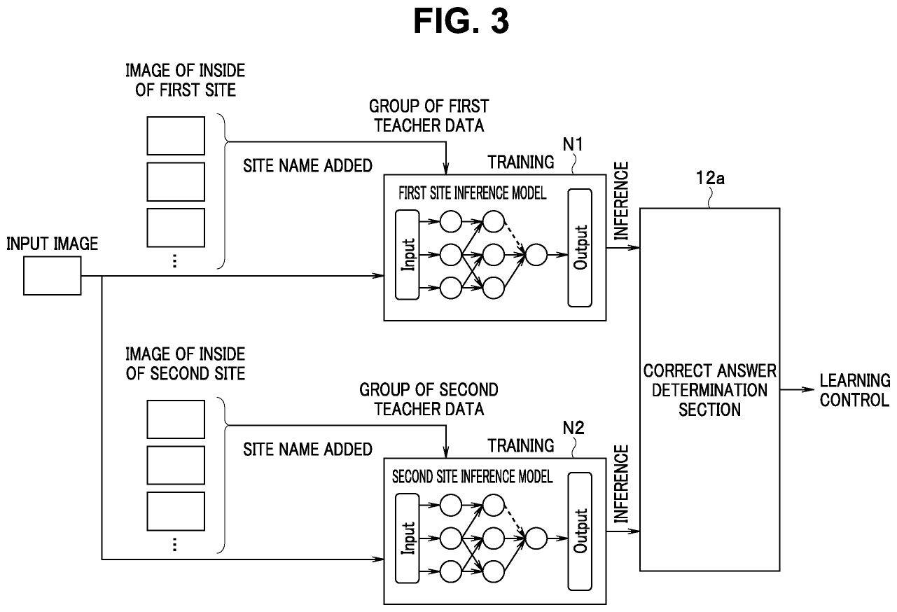

FIG. 3 is an explanatory diagram for explaining an inference model for enabling site determination. When determining the sites by the AI processing, the site determination section 12 adopts a network corresponding to each of the sites. The example in FIG. 3 shows two networks N1 and N2 for inferring a first site and a second site. A large number of images corresponding to inputs and outputs are provided as teacher data (hereinafter also referred to as training data) to each of the networks N1 and N2. In the example of FIG. 3, a plurality of images (quadrangular frames) of an inside of the first site are inputted, as the training data, to the network N1, and a plurality of images (quadrangular frames) of an inside of the second site are inputted, as the training data, to the network N2. The images to be inputted are added with, for example, site names corresponding to the respective images, as annotations.

Network designs of the networks N1 and N2 are decided such that the networks N1 and N2 can each obtain outputs corresponding to inputs by being trained with the large amount of training data. In other words, in the example of FIG. 3, when the images of the inside of the first site are inputted, information of the site name of the first site is outputted from the network N1, and when the images of the inside of the second site are inputted, information of the site name of the second site is outputted from the network N2. Thus, an inference model for detecting the site name of the first site from the input images is constructed by the network N1, and an inference model for detecting the site name of the second site from the input images is constructed by the network N2.

A correct answer determination section 12a determines whether the inference results from the networks N1 and N2 are correct answers, and when the inference results are incorrect, the correct answer determination section 12a performs learning control for reconstructing the network designs of the networks N1 and N2.

Note that “deep learning” has a structure formed by multilayering a process of “machine learning” using neural networks. A representative example is “feedforward neural network” in which information is fed from front to back to make determinations. The simplest structure of such a neural network may include only three layers, that is, an input layer composed of m1 neurons, an intermediate layer composed of m2 neurons defined by parameters, and an output layer composed of m3 neurons corresponding to the number of classes to be distinguished. Then, the respective neurons in the input layer and the intermediate layers are connected with connection weights, and the respective neurons in the intermediate layer and the output layer are connected with connection weights, and the intermediate layer and the output layer are added with bias values, which results in easy formation of a logic gate. Although the three layers may be enough for simple distinguishment, if the number of the intermediate layers is increased greatly, learning a method of combining a plurality of feature values would be possible in the process of machine learning. In recent years, the neural networks including 9 to 152 layers are practical in view of time required for learning, determination accuracy, and energy consumption.

Various known networks may be used as the network N1 adopted for machine learning. For example, regions with CNN features (R-CNN), fully convolutional networks (FCN), etc., that use a convolutional neural network (CNN) may be used. These networks include processing called “convolution” for compressing the feature values of the images, operate with minimal processing, and are excellent in pattern recognition. In addition, to handle more complex information and to cope with information analysis in which the meaning changes depending on the sequence or order, a “recurrent neural network (fully connected recurrent neural network)” in which information flows bi-directionally may be used.

In order to achieve these technologies, conventional general-purpose arithmetic processing circuits such as the CPU, the FPGA, and the like may be used. However, in view of the fact that a large part of the processing performed by the neural network is matrix multiplication, a processing unit called a GPU or a Tensor Processing Unit (TPU), which is specialized for the matrix calculation, is used in some cases. In recent years, a “neural network processing unit (NPU)”, which is hardware dedicated for artificial intelligence (AI), is designed to be able to be integrated and incorporated with another circuit such as the CPU, and in some cases, the NPU is a part of the processing circuit.

In addition, not only the deep learning, but methods of known various kinds of machine learning may be adopted to acquire the inference model. For example, there are methods such as a support vector machine and a support vector regression. The learning referred to here is for calculating weights of an identifier, filter coefficients, and offsets, and in addition, there is a method of using logistic regression processing. To cause a machine to determine something, humans have to teach the machine how to determine. In this practical example, the method has been adopted in which the determination of the images is derived through the machine learning. However, it is also possible to apply and use a rule-based method in which a specific judgment is made using rules which are obtained by humans through experiences and heuristics.

In the present embodiment, for each of the sites determined by the site determination section 12, features of each of the sites (hereinafter, referred to as a site-specific features) are determined, and classification is performed according to the determination results. Determination items for determining the site-specific features include a presence or absence of a diseased part, passage time, a number of acquired frames, a number of times of losing sight of a lumen direction, a number of times of insertion/withdrawal direction change, a color feature, a deformation during gas-feeding, a residue degree, a length, a thickness, a color, wrinkles, a movement, an elasticity, twisting, sagging, etc.

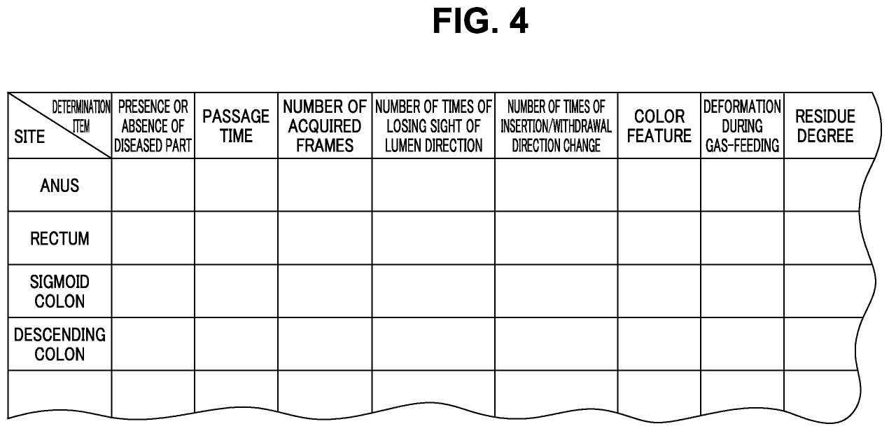

FIG. 4 is an explanatory diagram showing an example of correspondences between the respective sites and the respective determination items by classifying them in a table format. As the examples of the sites, the anus, the rectum, the sigmoid colon, and the descending colon are shown.

For the determination item of the “presence or absence of the diseased part”, a size, a position, a number, a color, etc., of the diseased part are determined. The determination items, the “passage time”, the “number of acquired frames”, the “number of times of losing sight of a lumen direction”, and the “number of times of insertion/withdrawal direction change” indicate a state of the insertion portion during the insertion and withdrawal of the insertion portion of the endoscope, and these items respectively indicate the time required for the insertion portion to pass through each of the sites, the number of image pickup frames of each of the sites, the number of times of losing sight of the lumen (deep part) direction during the image pickup of each of the sites, and the number of times of the insertion and withdrawal of the insertion portion during the image pickup of each of the sites. The “color feature” indicates features such as the color and the change in the color of each of the sites, and the “deformation during gas-feeding” indicates the feature of the state where the shape of each of the sites changes by gas-feeding to each of the sites. Based on the appearance of deformation, such as a bulge of tissues of the lumen during the gas-feeding, the features of the tissues, for example, whether they are elastic, hard, or soft, can be known. Substantially the same is true on liquid-feeding, and features, such as the effects of the liquid-feeding, may be recorded. Thus, the image classification device 10 in the present application is characterized in that a determination item database (DB) 40 for referring to the determination items showing the features to be determined for the above-described sites, contains the features (determination items) for determining the site-specific features in the image changes according to active treatment. The active treatment indicates not only the normal screening and the observation for diagnosis, but also actions (liquid-feeding, gas-feeding, and irradiation of special light (and image determination at the time), dyeing (and image determination at the time), and an action using a treatment instrument) which are different from those in the normal observation and performed actively on an object.

In addition, the “residue degree” indicates the degree of the amount of residual substances such as feces. Needless to say, if the features of the residual substances such as the color, the size, the position where the residual substances are left are specifically described or recorded, the features may possibly be used for specifying the features of internal structures, the digestive capability, and the diseases of the digestive organs.

In addition to the gas-feeding, the feature determination is also possible as to whether cleaning by liquid-feeding is effective. To put it differently, it can be said that reactions to measures that actively work on the lumen (active operation) are classified as the features. Whether or not these active operations are performed differs and the application sites of these active operations differ in each case. Therefore, comparison between the cases may not be possible. In view of this, an examination item, for example, “perform gas-feeding to this site to confirm a state of softness of a lumen”, may be included in the determination item DB 40, and during an examination, a guide for urging an examiner to perform the operation regardless of cases may be provided and the examination item may be set as a standardized one.

When the endoscope is directly connected to the image classification device 10 to perform an endoscopy, recommendation information regarding the active operation recommended for each of the anatomical sites may be recorded in the determination item DB 40, and according to the recommendation information, a guide for the endoscope operator may be displayed in accordance with a display device on which the endoscopic images are displayed. The display control section 16 is configured to include such a synthetic display function.

The determination items, the “length”, the “thickness”, the “color”, the “wrinkles”, the “movement”, the “elasticity”, the “twisting”, and the “sagging” respectively indicate the features of the length, the thickness, the color, the wrinkles, the movement, the elasticity, the twisting, and the sagging of each of the sites. In a case of determining the length and the like, a method of determining the insertion/withdrawal time, or determining the microstructures such as the blood vessels as patterns, to determine how long the patterns continue based on the images may be used. A method in which the inference model is used to infer the length of the lumen from the endoscopic images may be employed. The inference model used here has been trained using, as the training data, the length of the lumen, which has been obtained based on the images acquired during the movement of the endoscope through the lumen and X-ray images. Also other features, such as the number, the size, the spacing of the folds in the intestinal tract may be recorded.

The site-specific feature determination section 13 performs determination on the site-specific features, which are the features of the site determined by the site determination section 12, for one or more determination items. In other words, the site-specific feature determination section 13, which is a feature determination section, performs determination on the site-specific features for each of the sites, for the various determination items, to obtain site-specific feature information. The site-specific feature determination section 13 may be configured to obtain the site-specific feature information that classifies and shows the determination results of the site-specific features in a text format or a table format. For example, the site-specific feature determination section 13 may perform determination of the respective determination items, by the image analysis or the AI processing on the input images. The site-specific feature determination section 13 may determine the site-specific features based on the image changes in the respective frames of the images that are sequentially obtained. As shown in FIG. 4, in some cases, common determination items are set for all of the sites, but the determination items may be different for each of the sites. In addition, information in a table format may be held for each of the sites for which the determination items are common. The determination item DB 40 is a database configured to store the information regarding the determination items for each of the sites. The site-specific feature determination section 13 reads out, for each of the sites, the determination items stored in the determination item DB 40, to perform determination for the read determination items. In other words, the image classification device 10 in the present disclosure may include the database for referring to the features (determination items) for determining the site-specific features for each of the sites, for a plurality of sites through which the endoscope passes.

The active operation determination section 14 determines the signal which is based on the active operation performed by an operator during the insertion of the endoscope into a body or the withdrawal of the endoscope from a body, to obtain active operation information. The active operation may include, for example, a photographing operation of still images by operation of the operation portion of the endoscope, an operation of the device for feeding gas or liquid into the body, etc. The active operation determination section 14 receives the operation signals based on the operations to the devices and the like, to obtain the active operation information as determination results of the active operations. Note that the active operation determination section 14 may be configured to obtain the determination results of the active operations by the image analysis performed on the input images. It sometimes can be known, from the changes in the images over time, that the liquid feeding (images of splashing of the liquid are obtained, and the splashing settles down later, etc.) and the gas-feeding (tissues swelled and returned to their original state, etc.) have been performed.

The associating section 15 records the site-specific features for each of the sites in association with the input images. In other words, the associating section 15 associates the respective determination results by the site determination section 12, the site-specific feature determination section 13, and the active operation determination section 14 with the input images. The associating section 15 controls the recording control section 18, so as to record the results of the association in a recording device 30. For example, the associating section 15 may record these respective determination results in association as metadata of the still image files or the moving image files. Accordingly, site information indicating that the image of which of the sites is picked up, the site-specific feature information for the site in a table format or a text format, for example, or the active operation information regarding the active operation performed at the photographing timing (capture timing) of each of the frames are recorded in association with the still image file or image information of each of the frames of the moving image file. Note that, also the accompanying information accompanying the input images are recorded as they are in the image file, in association as the meta data. In addition, when the site determination section 12 determines each of the sites by the AI processing, the information regarding the inference model used for the determination may also be recorded in association with the images.

The recording control section 18 controls recording of the data, according to the associating section 15. The recording device 30 includes a recording medium, not shown, and records the image files subjected to the association, under the control by the recording control section 18. Thus, the images of many persons undergone the endoscopies are recorded in association not only with the accompanying information added at the image pickup stage, but also with, for each of the frames, the site information, the site-specific feature information, and the active operation information.

The display control section 16 performs control related to display of a display device 20. The display device 20 is a display device such as a liquid crystal display (LCD), etc., and performs various kinds of display under the control by the display control section 16. The display control section 16 can display, on a display screen thereof, a search result obtained by the search section 17 to be described later.

The search section 17 searches images based on a user operation performed on an input device, not shown. For example, the search section 17 may search the images in which both the site information and the site-specific feature information are common. In other words, the search section 17 can perform search processing based on the site-specific feature information, for the picked-up images of the site determined by the site determination section 12, to extract the picked-up images of a similar case. In addition, the search section 17 may search the images picked up at different times, for the same site of the same person. The search section 17 gives the search results to the display control section 16, and the display control section 16 causes the display device 20 to display the search results on the display screen thereof.

FIG. 1 shows a display example on the display screen of the display device 20. In the example shown in FIG. 1, an image Pe1 of a given site of an examinee α acquired currently (year of 2023) is displayed. In the image Pe1, an image Pe1a of a lesion part such as a polyp is displayed. The search section 17 searches images including the site information and the site-specific feature information that match the site information and the site-specific feature information that are associated with the image Pe1. For example, it is supposed that the site determination section 12 determines that the site in the image Pe1 is a large intestine.

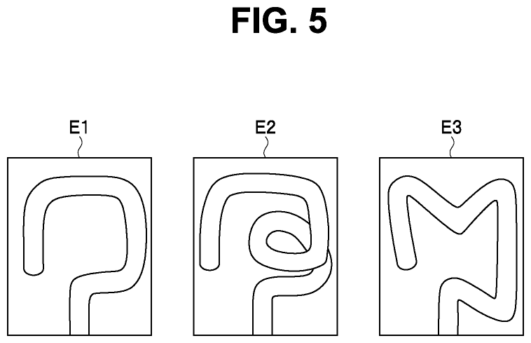

FIG. 5 is an explanatory diagram showing examples of the shape of the large intestine. An example E1 shows an example of the large intestine with little twisting and sagging, and an example E2 shows an example of the large intestine with great twisting, and an example E3 shows an example of the large intestine with great sagging. The site-specific feature information includes information of such shape features of the site. Even if the site is the same, organs that have different sizes, shapes, or the like may possibly differ in symptoms even when similar lesion parts exist therein. In view of this, in the present embodiment, even if the site is the same, the images including substantially the same site-specific features are the targets for the search.

The search section 17 searches the images including the site information and the site-specific feature information that correspond to those of the image Pe1. In other words, the images of the same site, i.e., the large intestine, that is, the images in which the shape, the size, and the elasticity of the site are similar, and a similar diseased part is included, in other words, the images including substantially the same site-specific features are obtained as the search results. Note that, depending on the method of classification of the site-specific features, the search section 17 may obtain, as the search results, not only the images including substantially the same site-specific features but also the images including similar site-specific features.

In the example shown in FIG. 1, an image Pe2 is searched and displayed, as the image including the site information and the site-specific feature information that correspond to those of the image Pe1. The image Pe2 includes an image Pe2a of a lesion part similar to that of the image Pe1a in the image Pe1. Note that the time when the image Pe2 was picked up is the year of 2020. Furthermore, the search section 17 searches whether progress information regarding contents of the treatment and the state of the diseased part of the person for whom the image Pe2 has been picked up exists, for example, the image obtained by picking up the image of the same site in the year of 2021 after the time when the image Pe2 was picked up exists, and displays the search result. An image Pe3 shows the image of this search result and includes an image Pe3a which shows that the lesion part shown in the image Pe2a has expanded.

In other words, the search section 17 uses the site-specific feature information obtained in a first case for each of the anatomical sites through which the endoscope passes in the process of inserting the endoscope into the body or in the process of withdrawing the endoscope from the body, to thereby be capable of searching for endoscopic images acquired in a second case.

According to the display in FIG. 1, for example, the physician can judge that the lesion part of the examinee α is likely to expand, based on the fact that, for a person who has the lesion part similar to that occurred in the large intestine of the examinee α and the site-specific features are substantially the same as those of the large intestine of the examinee α, the lesion part of the person expanded later. In addition, a patient can be identified based on the site-specific feature information. Therefore, even when patient information such as a patient name cannot be obtained, for example, using the site-specific feature information enables identification of the patient, recording of the case, etc.

### (Operation)

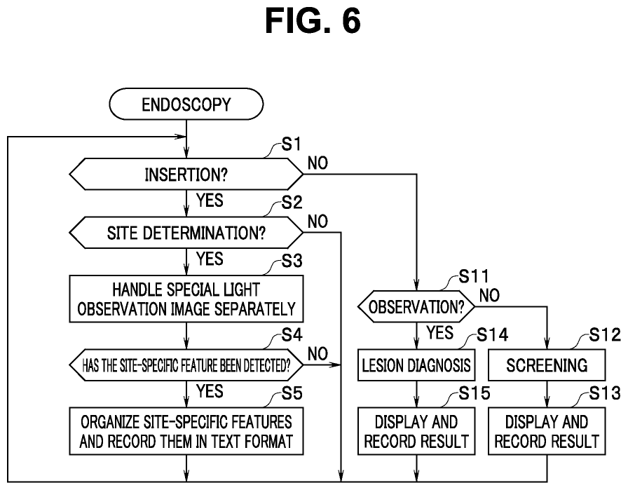

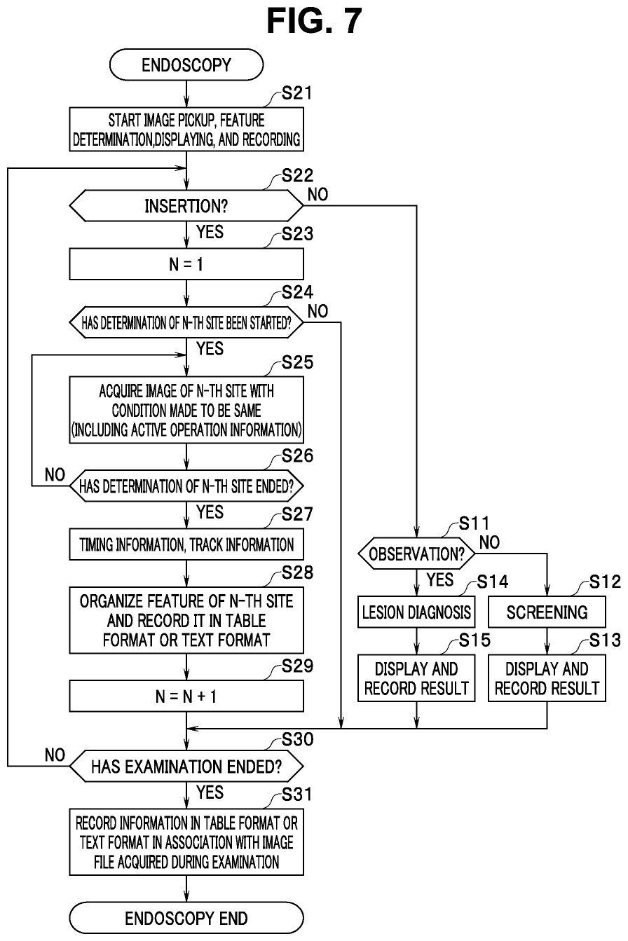

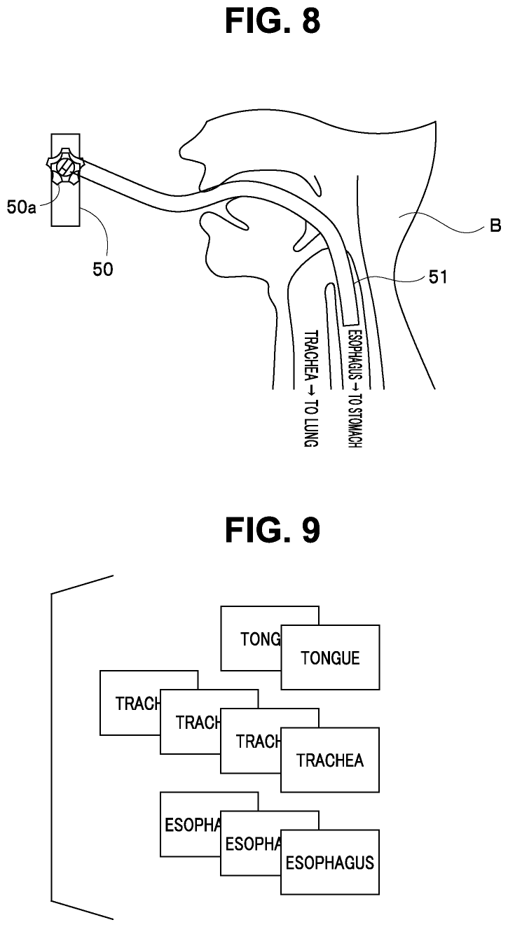

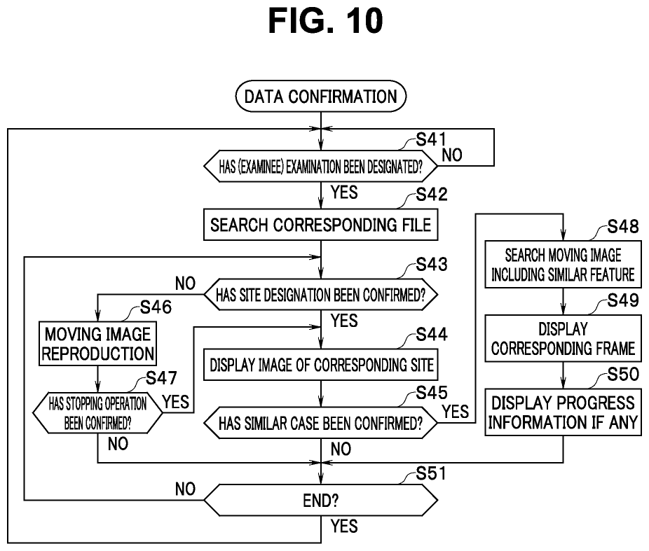

Next, description will be made on an operation of the embodiment thus configured, with reference to FIGS. 6 to 10. FIG. 6, FIG. 7, and FIG. 10 are flowcharts for explaining the operation of the embodiment. In addition, FIG. 8 is an explanatory diagram for explaining an example of an endoscopy. FIG. 9 is an explanatory diagram for explaining the images acquired in the example in FIG. 8.

FIG. 8 shows an oral cavity, a larynx, and a pharynx part of a human body B. FIG. 8 shows an appearance of an upper gastrointestinal endoscopy and a bronchial examination through a peroral endoscopy in which the insertion portion 51 is inserted from the mouth. As shown in FIG. 8, in an examination or treatment using an endoscope 50, the physician operates the endoscope 50, to insert an insertion portion 51 of the endoscope 50 into the human body B. A bending portion, not shown, is provided at a distal end of the insertion portion 51, and the physician operates a bending knob 50a, and the like, provided to the endoscope 50, to bend the bending portion and advances or retract the insertion portion 51, to insert the distal end of the insertion portion 51 up to an observation target site.

FIG. 9 shows an example of the images acquired in the endoscopy shown in FIG. 8. The quadrangular frames in FIG. 9 indicate the frames that are acquired, and during the insertion of the endoscope, a large number of images such as images of the tongue, the trachea, the esophagus, etc., are acquired in addition to the images of the observation target site. Thus, the insertion portion 51 passes through various sites before reaching the esophagus, and the images of the various sites are acquired.

During the insertion of the endoscope, the images acquired before reaching a specific observation target site are not used for diagnosis and the like, in some cases. However, during the insertion of the endoscope in which the endoscope passes through the respective sites in the process of reaching the observation target site, or during the withdrawal of the endoscope, the images of the respective sites other than those of the observation target site are also acquired. In other words, in addition to the endoscopic images to be used for diagnosis, a large number of endoscopic images are acquired in the process of insertion or withdrawal of the endoscope. In addition, such endoscopic images may possibly include information extremely useful for the next examination, and for the medical treatment and the like of another person who has similar symptoms.

However, even if these endoscopic images are just recorded, it would be extremely difficult to extract the useful information from the enormous numbers of images. In view of the above, in the present embodiment, the site-specific features are determined for each of the sites, and the determination results are recorded in association with the images as metadata, thereby enabling easy extraction of the useful information.

Although FIG. 6 shows an example in which the determination of such site-specific features is performed during the insertion of the endoscope, the determination may be performed during the screening or the observation. Alternatively, after the withdrawal of the endoscope, the endoscopic images recorded in the recording device, not shown, may be given to the image classification device 10, to perform the determination of the site-specific features.

In the example in FIG. 6, the image classification device 10 determines in S1 whether the endoscope is being inserted. During the insertion (YES in S1), the site determination section 12 initiates a site determination process, and determines whether the site determination has been performed (S2). When the site determination has been performed, in the next S3, after setting the special light observation images so as to be handled separately, the site-specific feature determination section 13 detects the site-specific features. In the special light observation, images of the organ illuminated with special light such as infrared light, etc., are picked up. In the determination of the site-specific features, a simple comparison between the picked-up images obtained by the image pickup of the organ illuminated with the normal light (white light) and the picked-up images obtained by the image pickup of the organ illuminated with the special light is impossible. Therefore, the special light observation images are handled separately. In other words, for the special light observation images, determination of the site-specific features is performed within a group of the special light observation images, and the special light observation images are used for a search within the group. Note that the image classification device 10 may determine whether the picked-up image is the special light observation image by image analysis, and when accompanying information indicating whether the picked-up image is the special light observation image is added thereto, the image classification device 10 may use the accompanying information to determine whether the picked-up image is the special light observation image.

Note that the site determination section 12 and the site-specific feature determination section 13 not only may perform the site determination and the site-specific feature determination by image analysis or the AI processing of the input images, but also may use the accompanying information and the active operation information to make these determinations. For example, for the deformation during the gas-feeding among the determination items for the site-specific features, information regarding the gas-feeding operation is acquired based on the active operation information, and the determination of the site-specific features is performed by the image analysis of the deformation of the organ during a period of the gas-feeding operation. In other words, the active operation information in this case relates to the timing of the picked-up image, and the determination of the site-specific features is performed according to the image change corresponding to the active operation, which enables effective determination results to be obtained. In addition, the site determination may be performed by using the accompanying information based on the output of the insertion shape observation device.

When the site-specific features are detected by the site-specific feature determination section 13 (YES in S4), the associating section 15 organizes the site-specific features for each of the sites, to record the site-specific features in association with the endoscopic image (S5). For example, the associating section 15 may record the site information and the site-specific feature information that are in the text format, in association with each of the frames as the meta data of the image. When the recording has ended, the procedure is returned to S1. Note that, also when NO determination is made in S2 or S4, the procedure is returned to S1. Note that during the recording in S5, not only the site information and the site-specific feature information, but also information such as the accompanying information included in the input image and the active operation information, and various pieces of information such as a type of an inference model used in the site determination are recorded.

Since the enormous pieces of image information obtained in the endoscopy are thus organized by each of the site-specific features, the site-specific feature information for each of the anatomical sites through which the endoscope passes in the process of insertion or withdrawal of the endoscope into or from the body in a specific case is used, to thereby enable a search of endoscopic images in another case.

When determining that the endoscope is not being inserted in S1 (NO in S1), the image classification device 10 determines, in S11, whether the endoscope is performing observation. When determining, in S11, that the endoscope is not performing observation (NO in S11), the image classification device 10 performs screening in S12. Note that the screening refers to the action of picking up images of a relatively large range to perform a search of a lesion part, for example. In addition, “observation” refers to, for example, the action of approaching the specific site to pick up the images thereof or zoom therein, and picking up detailed images of the specific site to confirm it.

The image classification device 10 can determine whether the endoscope is being inserted, performing screening, or performing observation, by the image analysis. For example, it is supposed that the insertion portion 51 advances through the inside of the lumen. In this case, an image part of a deep part of the lumen (deep part in a length direction of the lumen) where the illumination light from the distal end portion of the endoscope does not reach shows a lumen cross-sectional shape (substantially circular shape in many cases) with low luminance, and when the insertion portion 51 advances through the inside of the lumen, continuous images are obtained in which the image part is located substantially at the center of the endoscopic image and in the periphery of the image, patterns of the lumen wall move toward the periphery of the image. In addition, in the state where the lumen cross-sectional shape part with low luminance, which indicates the deep part of the lumen, is located at the center of the image, when the distal end of the endoscope is operated to be bent toward the wall surface of the object, the low-luminance part located at the center moves to the periphery of the screen. Therefore, such a state can be determined by the image analysis. Furthermore, in the case where a site having specific patterns is observed, for a long time, while getting close to or away from the site or while changing a viewing direction, the continuous images capture substantially the same patterns such as the blood vessels and the unevenness of the lesion. Therefore, the change in these patterns enables the observation state to be determined.

In S13, the result of the screening is displayed on the display device 20, and recorded in the recording device 30. In addition, during the observation (YES in S11), the image classification device 10 performs determination and discrimination of the lesion by known image processing, the artificial intelligence (AI) processing, or the like (S14), displays the results of the determination and discrimination on the display device 20, and records the results in the recording device 30 (S15). After the processing in S13 and S15, the procedure is returned to S1.

Thus, in the recording device 30, the image files, which are the endoscopic images of many persons undergone the endoscopies and in which the information such as the site information, the site-specific feature information, the accompanying information, the active operation information, etc., are associated with the images and added, are accumulated. The image information recorded in the recording device 30 can be classified by the information associated with each of the images, and based on the information associated with each of the images, the search of the images can be performed.

### (Moving Image)

The flow in FIG. 7 corresponds to the flow in FIG. 6, and shows an example in the case where the site-specific features are detected using the moving image. In FIG. 7, substantially the same processing steps as those in the flow in FIG. 6 are attached with the same reference signs and descriptions thereof will be omitted. Note that also the flow of FIG. 7 shows the example in which the site determination and the determination of the site-specific features are performed during the insertion of the endoscope. However, the site determination and the determination of the site-specific features may be performed during the withdrawal of the endoscope, during the observation by the endoscope, during the screening, or after the end of the endoscopy.

In S21, the image pickup by the endoscope starts, determinations of the site and the site-specific features (feature determination) are started, and displaying and recording of the picked-up image are started. In S22, the image classification device 10 determines whether the endoscope is being inserted. During the insertion (YES in S22), a variable N indicating a site is initialized to 1, and a start of determination of an N-th site is judged (S24). When the determination of the N-th site is started (YES in S24), the image classification device 10 acquires the image of the N-th site, in S25, with the image pickup conditions made to be substantially the same, that is, under the special light observation or the normal light observation, for example. Note that the accompanying information and the active operation information are also acquired simultaneously as the image.

The site determination section 12 determines each of the sites using the acquired image, the accompanying information, and the active operation information. Furthermore, in S26, the site-specific feature determination section 13 determines the site-specific features for each of the sites using the acquired image, the accompanying information, and the active operation information. The image classification device 10 determines in S26 whether the determination processing for the N-th site has ended. When the determination of the N-th site has not ended (NO in S26), the procedure is returned to S25, and determinations of the site and site-specific features are continued.

When the determination of the N-th site has ended (YES in S26), the image classification device 10 acquires timing information and track information in S27. The timing information is information regarding the image pickup time (image capture timing) for the N-th site, and the track information is information indicating a position of the recording unit (track) in the image information of the N-th site.

The associating section 15 organizes the site-specific feature information for the N-th site, and temporarily records the site-specific feature information in a table format or a text format in the image information as metadata (S28). Note that the site-specific feature information is made to correspond to the timing information and the track information, and is recorded in association with the endoscopic image. Using the timing information and the track information enables the site-specific feature information of the N-th site to be extracted easily from the recorded image information. Here, when the meta data is recorded in the text format, since the number of the patterns of the texts to be recorded can be counted in advance, codes are added so as to correspond respectively to the patterns of the respective texts, and the codes may be recorded as the meta data. In this case, information regarding the correspondence between the patterns of the texts and the codes is shared, as appropriate, with the system that refers to the codes.

The image classification device 10 increments the variable N indicating the site in S29, and determines whether the examination has ended in S30. When the examination has not ended (NO in S30), the procedure is returned to S22, and processing steps are repeated. When the image pickup has been performed for all the supposed sites, and the examination has ended (YES in S30), the associating section 15, in S31, controls the recording control section 18 to cause the recording device 30 to record the site-specific feature information for all the sites in the table format or the text format for each of the sites, as the meta data, in the image file (image files acquired during the examination), and the examination ends. With this, the recording control section 18 records the information in the table format, as the site-specific feature information describing the features of the respective sites, for the respective plurality of sites through which the endoscope passes in the insertion or withdrawal process.

Note that the operation in the case of NO determination in S22 is substantially the same as that in FIG. 6.

Thus, in the recording device 30, the image files, which are the endoscopic images of many persons undergone the endoscopies and in which the information such as the site information, the site-specific feature information, the accompanying information, and the active operation information are associated with the images and added, are accumulated. The image information recorded in the recording device 30 can be classified by the information associated with each of the images, and based on the information associated with each of the images, the search of the images can be performed.

### (Use of Classified and Accumulated Information)

FIG. 10 shows a using method (data confirmation flow) of the image information recorded in the recording device 30.

The image classification device 10 receives, through an input device, not shown, examination designation for designating the image information to be used. The image classification device 10 is in a waiting state of an examination designation of a certain examinee in S41. For example, a user can designate target image information by designating a person's name, an examination date and time, a type of the examination such as an upper endoscopy, and the like. When the examination designation is made (YES in S41), the search section 17 searches the image file corresponding to the designated examination (S42). The search section 17 gives the image file as the search result to the display control section 16, to cause the display device 20 to display the image.

The search section 17 determines whether designation of the site (site designation) is made for the site, images of which the user desires to confirm (S43). When the site designation is not made, the search section 17 controls the display control section 16, to start moving image reproduction of the image which is being displayed (S46), and then determines whether a reproduction stopping operation is made (S47). In S47, the search section 17 continues the moving image reproduction until the stopping operation is made by the user, and when the stopping operation is performed, moves the procedure to S44, and when the stopping operation is not performed, moves the procedure to S51. In S51, the search section 17 determines whether the procedure has ended, and when the procedure has not ended, returns the procedure to S43.

When the stopping operation is performed in the middle of the moving image reproduction (YES in S47), the search section 17 then causes the display device 20 to display the image of the site, which is being displayed at the time when the stopping operation is performed, as a still image (S44). In addition, in S43, when the user performs an input for designating the site such as the stomach or the large intestine through the input device, not shown, (YES in S43), the search section 17 then causes the display device 20 to display the image of the site according to the designation by the input as a still image (S44).

Thus, the image, which is the image of the designated examinee and is the image of the site which the physician, or the like, desires to confirm, is displayed. The search section 17 determines, in S45, whether an operation for confirming a similar case has been performed. When the operation for confirming is not performed (NO in S45), the procedure is moved to S51, and when the operation for confirming is performed, the procedure is moved to S48.

In S48, the search section 17 searches for a moving image including similar features. In other words, the search section 17 searches the enormous pieces of image information accumulated in the recording device 30 to retrieve the image part in the image including the site based on the site information and the site-specific features based on the site-specific feature information (S48), reads out the image part based on the timing information and the track information that designate the position of the image part, and causes the display device 20 to display the image part as a still image (S49) (see the image Pe2 in FIG. 1). Note that the search section 17 may use the accompanying information and the active operation information for the search processing.

Further, when the progress information exists for the examinee for whom the similar image searched in S48 was picked up, the search section 17 searches for and retrieves the progress information, and then causes the display device 20 to display the retrieved progress information (see the image Pe3 in FIG. 1).

Thus, in the present embodiment, the respective sites are classified by the site-specific features based on the images of the respective sites, and the classification results are accumulated, and the images are searched for based on the site-specific features, which enables the images including similar site-specific features to be searched for easily. Such a configuration enables easy extraction of not only a certain patient's own examination images including similar site-specific features but also another patient's examination images similar to the certain patient's examination images, which can be used for diagnosis and prevention.

Note that the images that include similar features including also the medical procedure may be searched for by using the information regarding the operation of the operator (physician who performs operation) and the operation of assistants, during the endoscopy, and during the treatment or surgery using the endoscope. Similarly, the images may be searched for using setting information of the treatment instrument and other instruments that are used in the endoscopy, the treatment, and the surgery.

In the present embodiment, description has been made by taking the gastrointestinal endoscopy as an example. However, the present embodiment can be applied also to a laparoscopic surgery, a thoracoscopic surgery, etc., and endoscopes (surgical fields, otolaryngology fields, gynecology fields, urological fields, etc.) other than the endoscope configured to be inserted into the digestive tracts. Although the tract such as the intestinal tract is a single continuous tract, the site such as a bronchus includes branched tracts. Therefore, in such a case, information regarding which of the tracts has been selected may be added.

In addition, since an endoscope, because of its characteristics, travels back and forth in a body when inserted and withdrawn, the features may be classified separately in each of the process of insertion and the process of withdrawal. In that case, two tables, each of which is like the table shown in FIG. 4, are created for one case. The features may possibly change by the lumen and the like being pushed or pulled due to friction. In view of this, the features may be organized as average values in the tables.

The present disclosure is not limited to the above-described embodiment as it is, and can be embodied by modifying the constituent elements within a range without departing from the gist of the disclosure at the practical stage. In addition, various disclosures can be achieved by appropriately combining the plurality of constituent elements disclosed in each of the above-described embodiments. Some of the constituent elements may be deleted from all the constituent elements shown in the embodiment, for example. Furthermore, constituent elements over different embodiments may be combined as appropriate.

Among the above-described technologies, most of the control and functions mainly described in the flowcharts can be set by a program, and the above-described control and functions can be implemented by the program being read and executed by a computer. The entirety or a part of the program can be recorded or stored as a computer program product in a portable medium such as a flexible disk, CD-ROM, a non-volatile memory, or the like, or a storage medium such as hard disk, a volatile memory, or the like. The program can be distributed or provided at the time of product shipment or through a portable medium or a communication line. It is possible for a user to easily implement the image classification device for the endoscope according to the present embodiment by downloading the program through a communication network to install the program into a computer, or installing the program from a recording medium into the computer.

### (General Interpretation Notes)

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another.

The term “processing circuitry,” as used herein, refers to any hardware or combination of hardware and software configured to execute the operations described. The term “processing circuitry” is a broad structural term that encompasses, without limitation, general-purpose processors (e.g., CPUs, GPUs), microcontrollers, digital signal processors (DSPs), application-specific integrated circuits (ASICs), field-programmable gate arrays (FPGAs), programmable logic devices (PLDs), and discrete logic circuits. In addition to logic or execution units, the processing circuitry may explicitly include or be integrally coupled to memory (e.g., registers, cache, RAM, ROM, or other storage media) that stores data, software, firmware, or instructions contributing to the processing operations. Accordingly, the processing circuitry may be implemented as a specialized hardware circuit having fixed logic, a programmable circuit executing instructions stored in an internal or external memory, or any combination thereof. The processing circuitry may be configured as, or include, one or more processors. Thus, the term “processor” used in the description of embodiments is to be understood as a specific example of the processing circuitry or as a component included within the processing circuitry. Furthermore, the processing circuitry may be distributed across multiple devices or locations (e.g., cloud computing) or consolidated within a single device. The term “processing circuitry” implies a concrete structure and is not intended to be construed as a purely functional “means” lacking structural support.

The term “non-transitory computer-readable (storage) medium” refers to any tangible device or medium capable of storing code or data for access by a computer or processing circuitry. This term encompasses a single storage medium or a group of multiple storage media, which may be locally or remotely distributed (e.g., across a network, in a cloud computing environment, or within a distributed ledger system) and may collectively store information in a coordinated or distributed manner. Examples of such media include, but are not limited to, non-volatile media (e.g., optical disks, magnetic disks, flash memory, ROM) and volatile media (e.g., dynamic memory, RAM, registers, buffers, and caches). Importantly, the term “non-transitory” is intended to exclude only transitory propagating signals per se (e.g., carrier waves, electromagnetic waves, or digital signals in transit through a transmission medium) and does not exclude statutory subject matter such as volatile memory where data is stored temporarily.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

In this specification and the appended claims, the singular forms “a,” “an,” and “the” include plural referents unless the context clearly dictates otherwise. For example, reference to “a recess” includes a single recess as well as a plurality of recesses.

Terms of degree, such as “substantially,” “about,” “approximately,” and “generally,” as used herein, represent a reasonable amount of deviation from the specified value or state such that the end result is not significantly changed. These terms should be construed as including a deviation of at least ±5% or ±10% of the modified value, or a deviation recognized by a person of ordinary skill in the art, unless otherwise indicated.

As used herein, geometric, spatial, or directional terms such as “parallel,” “perpendicular,” “orthogonal,” “intersecting,” “polygon,” or specific angles (e.g., “45 degrees”) are not intended to require strict mathematical or geometric exactness. Rather, these terms should be understood to encompass substantial approximations, manufacturing tolerances, and practical variations recognized by those skilled in the art, unless otherwise explicitly claimed.

In the present disclosure, terms such as “same shape,” “uniform shape,” or “common shape” refer to having the same general profile, contour, or topological structure (e.g., both being T-shaped), and do not necessarily require identical dimensions, sizes, scales, or proportions, unless explicitly stated otherwise.

Non-limiting examples according to aspects of the present disclosure will be described in the following clauses:

Clause 1: An image classification device for an endoscope, comprising:
a site determination section that determines anatomical sites in a human body through which the endoscope has passed, according to picked-up images obtained sequentially in a process of insertion of the endoscope into a body or in a process of withdrawal of the endoscope from the body;
a feature determination section that determines a site-specific feature, which is a feature of the site for each of the determined sites, for one or more determination items, and obtains anatomical site-specific feature information for each of the sites; and
an associating section that records the anatomical site-specific feature information for each of the sites in association with the picked-up images.

Clause 2: The image classification device according to clause 1, further comprising:
a recording control section that records information in a table format, for each of a plurality of sites through which the endoscope has passed in the process of insertion or the process of withdrawal, as the anatomical site-specific feature information describing a feature of each of the plurality of sites.

Clause 3: The image classification device according to clause 1, further comprising:
a database for referring to the one or more determination items for determining the anatomical site-specific feature for each of a plurality of sites through which the endoscope has passed in the process of insertion or the process of withdrawal.

Clause 4: The image classification device according to clause 3,
wherein the database includes the one or more determination items for determining the site-specific feature in an image change of the picked-up images according to active treatment.

Clause 5: The image classification device according to clause 3,
wherein the database includes recommendation information regarding an active operation recommended for each of the sites, and
wherein the image classification device further includes a display control section that displays a guide for an endoscope operator according to the recommendation information.

Clause 6: The image classification device according to clause 1, further comprising:
a search section that uses the anatomical site-specific feature information obtained for each of the sites in a first case to search for an endoscopic image obtained in a second case.

Clause 7: The image classification device according to clause 1, further comprising:
an active operation determination section that determines a signal based on an active operation by an endoscope operator when the endoscope is being inserted into the body, and obtains active operation information,
wherein the associating section records site information indicating the site, the anatomical site-specific feature information, and the active operation information, in association with the picked-up images.

Clause 8: The image classification device according to clause 7,
wherein the picked-up images are added with accompanying information, and
wherein the associating section records the site information, the anatomical site-specific feature information, the active operation information, and the accompanying information, in association with the picked-up images.

Clause 9: The image classification device according to clause 1,
wherein the feature determination section obtains the anatomical site-specific feature information that classifies and shows a determination result of the site-specific feature in a text format, a code, or a table format.

Clause 10: The image classification device according to clause 9,
wherein the associating section, when recording a file of the picked-up images, records the file by adding the anatomical site-specific feature information in the table format to the picked-up images as metadata.

Clause 11: The image classification device according to clause 9,
wherein the associating section records the anatomical site-specific feature information in association with the picked-up images, so as to correspond to timing information of the picked-up images.

Clause 12: The image classification device according to clause 1,
wherein the feature determination section determines the site-specific feature based on an image change in each of frames of the sequentially obtained picked-up images.

Clause 13: The image classification device according to clause 7,
wherein the active operation information related to timings of the picked-up images is acquired and the site-specific feature is determined according to an image change of the picked-up images corresponding to the active operation.

Clause 14: The image classification device according to clause 1, further comprising:
a search section that performs search processing based on the anatomical site-specific feature information for the picked-up images of the site determined by the site determination section, and extracts picked-up images in a similar case.

Clause 15: The image classification device according to clause 14, further comprising,
a display control section that causes a display device to display a search result by the search section.

Clause 16: The image classification device according to clause 15,
wherein when progress information exists for an examinee for whom picked-up images in the similar case are obtained, the search section searches for the progress information, and
wherein the display control section causes the display device to display the progress information.

Clause 17: An image classification method comprising:
determining a site of anatomical sites in a human body through which the endoscope has passed, according to picked-up images obtained sequentially in a process of insertion of an endoscope into a body or in a process of withdrawal of the endoscope from the body;
determining a site-specific feature, which is a feature of the determined site, for one or more determination items, and obtaining anatomical site-specific feature information; and
recording the anatomical site-specific feature information for each of the sites in association with the picked-up images.

Clause 18: An image classification program for causing a computer to execute a procedure of:
determining a site of anatomical sites in a human body through which the endoscope has passed, according to picked-up images obtained sequentially in a process of insertion of an endoscope into a body or in a process of withdrawal of the endoscope from the body;
determining a site-specific feature, which is a feature of the determined site, for one or more determination items, and obtaining anatomical site-specific feature information; and
recording the anatomical site-specific feature information for each of the sites in association with the picked-up images.

Clause 19: An image classification method comprising:
determining a plurality of anatomical sites in a human body through which the endoscope has passed, according to picked-up images obtained sequentially in a process of insertion of an endoscope into a body or in a process of withdrawal of the endoscope from the body;
referring to a database in which one or more determination items indicating a feature to be obtained for each of the plurality of sites are organized, in order to obtain, for each of the plurality of sites, a site-specific feature which is a feature of each of the plurality of sites; and
determining the site-specific feature for each of the plurality of determined sites and obtaining anatomical site-specific feature information based on the site-specific feature such that the determined site-specific feature can be recorded in association with the picked-up images.

Clause 20: A search method comprising:
using anatomical site-specific feature information indicating a feature of each of anatomical sites through which an endoscope has passed in a process of insertion of the endoscope into the body or in a process of withdrawal of the endoscope from the body in a first case, to search for an endoscopic image obtained in a second case.

What is claimed is:

1. An image classification device for an endoscope, comprising:
a connection interface; and
processing circuitry configured to:
obtain, via the connection interface, images sequentially captured in a process of insertion of the endoscope into a body of a subject or withdrawal of the endoscope from the body;
determine anatomical sites in the body through which the endoscope has passed, according to the obtained images;
determine site-specific features of each of the determined anatomical sites for one or more determination items, thereby obtaining anatomical site-specific feature information for each of the determined anatomical sites; and
record the anatomical site-specific feature information for each of the determined anatomical sites in association with the obtained images.

2. The image classification device according to claim 1,
wherein the processing circuitry is further configured to record, in a table format, the anatomical site-specific feature information describing the site-specific features for each of the determined anatomical sites through which the endoscope has passed in the process of insertion or withdrawal.

3. The image classification device according to claim 1, further comprising a database configured to store information regarding the one or more determination items,
wherein the processing circuitry is further configured to refer to the database to determine the site-specific features for each of the determined anatomical sites through which the endoscope has passed in the process of insertion or withdrawal.

4. The image classification device according to claim 3,
wherein the information stored in the database includes at least one of the one or more determination items that is used to determine the site-specific features based on an image change of the obtained images resulting from an active treatment.

5. The image classification device according to claim 3,
wherein the information stored in the database includes recommendation information regarding an active operation recommended for each of the determined anatomical sites, and
wherein the processing circuitry is further configured to control a display device to display a guide for an endoscope operator according to the recommendation information.

6. The image classification device according to claim 1,
wherein the processing circuitry is further configured to use the anatomical site-specific feature information obtained for each of the determined anatomical sites in a first case to search for endoscopic images obtained in a second case.

7. The image classification device according to claim 1,
wherein the processing circuitry is further configured to:
determine a signal based on an active operation while the endoscope is inserted into the body to obtain active operation information; and
in recording the anatomical site-specific feature information, further record the obtained active operation information and site information indicating the determined anatomical sites in association with the obtained images.

8. The image classification device according to claim 7,
wherein the obtained images include accompanying information added thereto, and
wherein the processing circuitry is further configured to, in recording the anatomical site-specific feature information, the obtained active operation information, and the site information, further record the accompanying information in association with the obtained images.

9. The image classification device according to claim 1,
wherein the obtained anatomical site-specific feature information indicates the determined site-specific features classified in a text format, a code, or a table format.

10. The image classification device according to claim 1,
wherein the processing circuitry is further configured to, in recording the anatomical site-specific feature information, add the anatomical site-specific feature information as metadata to a file of the obtained images to be recorded.

11. The image classification device according to claim 1,
wherein the processing circuitry is further configured to, in recording the anatomical site-specific feature information, record the anatomical site-specific feature information in correspondence with timing information regarding respective capture timings of the obtained images.

12. The image classification device according to claim 1,
wherein the processing circuitry is further configured to, in determining the site-specific features, determine the site-specific features based on an image change between sequentially captured frames of the obtained images.

13. The image classification device according to claim 7,
wherein the processing circuitry is further configured to:
in obtaining the active operation information, obtain the active operation information related to respective capture timings of the obtained images; and
in determining the site-specific features, determine the site-specific features based on an image change of the obtained images corresponding to the active operation.

14. The image classification device according to claim 1,
wherein the processing circuitry is further configured to search the obtained images of the determined anatomical sites based on the anatomical site-specific feature information to extract images of similar cases.

15. The image classification device according to claim 14,
wherein the processing circuitry is further configured to control a display device to display the extracted images.

16. The image classification device according to claim 15,
wherein the processing circuitry is further configured to:
in searching the obtained images, search for and retrieve progress information of examinees for whom the extracted images were captured; and
in controlling the display device, control the display device to display the retrieved progress information.

17. The image classification device according to claim 1,
wherein the processing circuitry is further configured to, in determining the site-specific features, determine the site-specific features based on at least one of:
(i) reference to a database storing information regarding the one or more determination items;
(ii) an image change between sequentially captured frames of the obtained images; or
(iii) an image change of the obtained images corresponding to an active operation.

18. A method implemented by processing circuitry, comprising:
obtaining images sequentially captured in a process of insertion of an endoscope into a body of a subject or withdrawal of the endoscope from the body;
determining anatomical sites in the body through which the endoscope has passed, according to the obtained images;
determining site-specific features of each of the determined anatomical sites for one or more determination items, thereby obtaining anatomical site-specific feature information for each of the determined anatomical sites; and
performing at least one of:
recording the anatomical site-specific feature information for each of the determined anatomical sites in association with the obtained images; or
searching for endoscopic images obtained in a second case using the anatomical site-specific feature information obtained for each of the determined anatomical sites in a first case.

19. The method according to claim 18,
wherein determining the site-specific features includes referring to a database that stores information regarding the one or more determination items indicating features to be obtained for each of the determined anatomical sites.

20. A non-transitory computer-readable storage medium storing computer-readable instructions configured to, when executed by processing circuitry, cause the processing circuitry to:
obtain images sequentially captured in a process of insertion of an endoscope into a body of a subject or withdrawal of the endoscope from the body;
determine anatomical sites in the body through which the endoscope has passed, according to the obtained images;
determine site-specific features of each of the determined anatomical sites for one or more determination items, thereby obtaining anatomical site-specific feature information for each of the determined anatomical sites; and
perform at least one of:
recording the anatomical site-specific feature information for each of the determined anatomical sites in association with the obtained images; or
searching for endoscopic images obtained in a second case using the anatomical site-specific feature information obtained for each of the determined anatomical sites in a first case.

21. The non-transitory computer-readable storage medium according to claim 20,
wherein determining the site-specific features includes referring to a database that stores information regarding the one or more determination items indicating features to be obtained for each of the determined anatomical sites.
